(19) United States
(12) Patent Application Publication
(10) Pub. No.: US 2003/0044777 A1
(43) Pub. Date: Mar. 6, 2003

(54) FLOWTHROUGH DEVICES FOR MULTIPLE DISCRETE BINDING REACTIONS

(76) Inventor: KENNETH L. BEATTIE, CROSSVILLE, TN (US)

Correspondence Address: BLANK ROME COMISKY & MCCAULEY, LLP, 900 17TH STREET, N.W., SUITE 1000, WASHINGTON, DC, 20006, US

(21) Appl. No.: 09/213,932

(22) Filed: Dec. 17, 1998

Related U.S. Application Data
(63) Continuation-in-part of application No. 09/063,356, filed on Apr. 21, 1998, now abandoned.
Application No. 09/063,356 is a division of application No. 08/631,751, filed on Apr. 10, 1996.
Application No. 08/631,751 is a continuation-in-part of application No. 08/141,969, filed on Oct. 28, 1993, now abandoned.

(30) Foreign Application Priority Data
Dec. 27, 1994 (US) PCT/US94/12282

(51) Int. Cl. C12Q 1/68; G01N 33/53; C12M 1/34; C12M 3/00; G01N 33/566; G01N 33/543; G01N 33/552; G01N 33/544

(52) U.S. Cl. 435/6; 435/7.8; 435/973; 435/287.2; 435/288.4; 436/501; 436/518; 436/527; 436/535; 436/809

(57) ABSTRACT

Devices and methods for conducting binding reactions are described. The devices comprise first and second surfaces with channels extending between them. Specific binding reagents are immobilized in discrete groups of the channels. Sample passing through the channels reacts with the binding reagents. Binding of the sample component to the binding reagent in different groups of channels is detected providing information about sample composition. The devices provide increased surface area and accelerated reactions kinetics compared with flat surfaces.

This application is a continuation-in-part of co-pending application Ser. No. 09/063,356, filed Apr. 28, 1998, which is a continuation of application Ser. No. 08/631,751 (filed Apr. 10, 1996), which is a continuation of PCT/US94/12282 with an international filing date of Oct. 27, 1994, which is a continuation-in-part of application Ser. No. 08/141,969 (filed Oct. 28, 1993), now abandoned. The specifications of application Ser. Nos. 09/063,356, 08/631,751 and 08/141,969 and PCT/US94/12282 are incorporated by reference herein in entirety.

### BACKGROUND OF THE INVENTION

Microfabrication technology has revolutionized the electronics industry and has enabled miniaturization and automation of manufacturing processes in numerous industries. The impact of microfabrication technology in biomedical research can be seen in the growing presence of microprocessor-controlled analytical instrumentation and robotics in the laboratory, which is particularly evident in laboratories engaged in high throughput genome mapping and sequencing. The Human Genome Project is only one example of a task whose economics would benefit greatly from microfabricated high-density and ultra-high density devices that can be broadly applied in genome mapping and sequencing. Other analytical applications also would greatly benefit from the ability to simultaneously carry out and/or monitor arrays of assays. Examples include: high-throughput screening for new pharmaceuticals and other chemical entities, toxicology screening, and gene expression screening and analysis, clinical assays, microbiological analysis, environmental testing, food and agricultural analysis, genetic screening, monitoring chemical and biological warfare agents, and process control. Each of these applications involves carrying out and monitoring a reaction where a binding reagent is contacted with a test sample, and the occurrence and extent of binding of the binding reagent with specific components (target moieties) within the test sample is measured in some form.

One widely used analytical procedure in genome mapping illustrative of such applications is hybridization of membrane-immobilized DNAs with labeled DNA probes. Robotic devices currently enable gridding of 10,000-15,000 different target DNAs onto a 12 cm×8 cm membrane. See for example, Drmanac et al. in Adams et al. (Eds.), Automated DNA Sequencing and Analysis, Academic Press, London, 1994 and Meier-Ewert et al. Science 361:375-376 (1993). Hybridization of DNA probes to such membranes has numerous applications in genome mapping, including generation of linearly ordered libraries, mapping of cloned genomic segments to specific chromosomes or mega YACs, cross connection of cloned sequences in cDNA and genomic libraries, and so forth.

Genosensors, or miniaturized “DNA chips” currently are being developed for hybridization analysis of DNA samples. DNA chips typically employ arrays of DNA probes tethered to flat surfaces to acquire a hybridization pattern reflecting the nucleotide sequence of the target DNA. See, for example, Fodor et al. Science, 251:767-773 (1991); Southern et al. Genomics 13:1008-1017 (1992); Eggers et al. Advances in DNA Sequencing Technology, SPIE Conference, Los Angeles, Calif. (1993); and Beattie et al. Clin. Chem. 39:719-722 (1993). Such devices also may be applied in carrying out and monitoring other binding reactions, such as antibody capture and receptor binding reactions.

However, a serious limitation to miniaturization of DNA hybridization arrays or other types of binding arrays on membranes or other two-dimensional surfaces is the quantity of binding reagent that can be present per unit cross sectional area. This parameter governs the yield of hybridized DNA (or bound target) and thus determines for a given detection sensitivity the minimum spot size for detecting a given target with a given reagent. For a two-dimensional surface, the amount of DNA or binding reagent is a function of the surface area.

One example of the use of arrayed binding reactions is for so-called “sequencing by hybridization” (SBH). Two formats commonly are used for SBH: “format 1” versions involve stepwise hybridization of different oligonucleotide probes with arrays of DNA samples gridded onto membranes; and “format 2” implementations involve hybridization of a single nucleic acid “target sequence” to an array of oligonucleotide probes tethered to a flat surface or immobilized within a thin gel matrix. The term “genosensor” heretofore has been applied to a form of SBH in which oligonucleotides are tethered to a surface in a two-dimensional array and serve as recognition elements for complementary sequences present in a nucleic acid “target” sequence. The genosensor concept further includes microfabricated devices in which microelectronic components are present in each test site, permitting rapid, addressable detection of hybridization across the array. Recent initiatives in SBH aim toward miniaturized, high density hybridization arrays.

Sequence-by-hybridization determinations, including use of arrays of oligonucleotides attached to a matrix or substrate, are described, for example, in Khrapko et al., J. DNA Sequencing and Mapping, 1:375-388 (1991); Drmanac et al., Electrophoresis 13:566-573 (1992); Meier-Ewert et al., Nature 361:375-376 (1993); Drmanac et al., Science 260:1649-1652 (1993); Southern et al., Genomics 13:1008-1017 (1992); and Saiki et al., Proc. Natl. Acad. Sci. USA 86:6230-6234 (1989). General strategies and methodologies for designing microfabricated devices useful in DNA sequencing by hybridization (SBH) are described in: Eggers et al., SPIE Proceedings Series, Advances in DNA Sequence Technology, Proceedings Preprint, The International Society for Optical Engineering, Jan. 21, 1993; Beattie et al., Clinical Chemistry 39:719-722 (1993); Lamture et al., Nucl. Acids Res. 22:2121-2124 (1994); and Eggers et al., Biotechniques 17:516-525 (1994).

Typically, microfabricated genosensor devices are characterized by a compact physical size and the density of components located on the device. Known microfabricated binding devices typically are rectangular wafer-type apparatuses with a surface area of approximate one cm², e.g., 1 cm×1 cm. The bounded regions on such devices are typically present in a density of 10²-10⁴ regions/cm², although the desirability of constructing apparatuses with much higher densities has been regarded as an important objective. See Eggers et al. and Beattie et al., loc. cit., for discussions of strategies for the construction of devices with higher densities for the bounded regions. As in membrane hybridization, the detection limit for hybridization on flat-surface genosensors is limited by the quantity of DNA that can be bound to a two dimensional area. Another limitation of these approaches is the fact that a flat surface design introduces a rate-limiting step in the hybridization reaction, i.e., diffusion of target molecules over relatively long distances before complementary probes are encountered on the surface.

It is apparent, therefore, that high density devices for detecting multiple binding reactions, having improved detection sensitivity are greatly to be desired. Devices for detecting multiple binding reactions of biomolecules, for example, hybridization reactions of nucleic acids are particularly desirable.

### SUMMARY OF THE INVENTION

It is therefore an object of the present invention to provide improved devices for detecting multiple binding reactions.

It is another object of the invention to provide methods of detecting multiple binding reactions using the devices.

In accomplishing these objects, there has been provided, in accordance with one aspect of the present invention, a flow-through device comprising a substrate containing first and second surfaces, having a multiplicity of discrete channels extending through the substrate from the first surface to the second surface, a first binding reagent immobilized in a first group of the channels, and a second binding reagent immobilized in a second group of the channels, where the groups of the channels define an array of a multiplicity of discrete and isolated regions arrayed across the substrate surface. A test sample is applied that penetrates through the substrate and a detector capable of identifying and addressing each of the discrete and isolated regions is used to determine and report whether a binding reaction has taken place in the regions. Detection of a binding reaction between the binding reagents in one or more of the discrete and isolated regions and a test sample provides information for identifying or otherwise characterizing molecular species in the test sample. In one embodiment, the first and second binding reagents differ from one another. In another embodiment, the first and second binding reagent bind different target molecules. In yet another embodiment, the binding reagent is immobilized on the channel walls of the substrate.

In further embodiments, the substrate further comprises a rigid support, where the rigid support is integral to the substrate, or is bonded to the substrate. In other embodiments, the rigid support is a manifold comprising wells for delivering fluids to groups of channels of the substrate.

In further embodiments, the substrate is fabricated from glass or silicon. In particular embodiments in this regard, the substrate is made of nanochannel glass or oriented array microporous silicon.

In one embodiment, the discrete channels may have diameters in ranges of from about 0.033 micrometers to about 10 micrometers, from about 0.05 to 0.5 micrometers, from 1 to 50 micrometers, from 10 to 100 micrometers, or from 50 to 250 micrometers. In other embodiments, the channels may have cross sectional areas in ranges of from between about 8.5×10⁻⁴ μm² to about 80 μm², from about 2×10⁻³ μm² to about 0.2 μm², from about 0.8 μm² to about 2000 μm², from about 80 μm² to about 8000 μm², or from about 2,000 μm² to about 50,000 μm². In further embodiments, the channels have diameters of from about 0.45 micrometers to about 10 micrometers.

In still another embodiment, the substrate is from about 100 μm to about 1000 μm thick. In other embodiments the substrate is from about 10 μm to about 250 μm, from about 50 to about 500 μm, from about 250 μm to about 1.5 mm, or from about 500 μm to about 2 mm thick. In yet another embodiment, the channels have an inner surface area of between about 10 μm² and about 3×10⁴ μm².

In a further embodiment, the groups of channels have areas of between about 20 μm² to about 3×10⁶ μm², and in a still further embodiment, there are between 400 and 4400 of said groups of discrete channels per cm² of cross-sectional area of the substrate.

In yet another embodiment, the inner surface area of the channels in a group of channels is from about 100 to about 1000 times the cross sectional area of the group of channels.

In accomplishing another goal of the invention there have been provided methods of using the device described above for carrying out binding reactions selected from one or more of the following group of binding reactions, involving small molecules, macromolecules, particles and cellular systems.

In particular embodiments, the binding reagents are effective for carrying out an analytical task selected from the group consisting of sequence analysis by hybridization, analysis of patterns of gene expression by hybridization of mRNA or cDNA to gene-specific probes, immunochemical analysis of protein mixtures, epitope mapping, assay of receptor-ligand interactions and profiling of cellular populations involving binding of cell surface molecules to specific ligands or receptors.

In further particular embodiments, the binding reagents are selected from the group consisting of DNA, proteins and ligands, and in a particular embodiment are oligonucleotide probes. The oligonucleotide probes may be attached to the channel surfaces via a primary amine group incorporated into the probes prior to immobilization. In a particular embodiment, the probes are attached to the channel surfaces via a terminal primary amine derivative of the polynucleotide and the glass substrate is derivatized with epoxysilane.

In yet another embodiment, binding reagents are fixed in the channels of the substrate by means of a spacer that allows optimal spacing between the substrate surface and the binding reagent, thereby allowing the most efficient interaction between the binding reagent and the molecules in the test sample. When oligonucleotides are attached to a glass substrate derivatized with epoxysilane using an oligonucleotide terminal primary amine derivative, the oligonucleotide-silane fixation may comprise the incorporation of one or more triethylene glycol phosphoryl units as spacers.

In other embodiments, the oligonucleotides are fixed in groups of channels that form isolated and discrete regions of the substrate by attaching a terminal bromoacetylated amine derivative of the oligonucleotide to a platinum or gold substrate derivatized with a dithioalkane.

In yet another embodiment, the test sample is applied to the channels of the device by flooding a surface of the substrate with the sample and placing the other surface of the substrate under negative pressure relative to the first surface, whereby the resulting vacuum facilitates the flow through the substrate.

In a still further embodiment, the test sample is applied to the channels of the device by flooding a surface of the substrate with the sample and placing that surface of the substrate under positive pressure relative to the second surface, whereby the resulting pressure facilitates the flow through the substrate.

In still another embodiment, the molecules in the test sample are identifiable by radioisotope, fluorescent, or chemilumineseent labels.

In a further embodiment, the binding reactions in the device may be detected by a charge-coupled device (CCD) employed to detect hybridization of radioisotope-fluorescent-, or chemiluminescent-labelled polynucleic acids.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A and 1B show a substrate containing the channels that comprise the binding region for the binding reagents fixed therein. The binding region is a microchannel or nanochannel glass wafer and is shown with an optional attached upper manifold, where the manifold layer contains an array of tapered wells that can be used as one method of applying different samples of binding reagents or test samples to particular groups of channels on the chip. For clarity, only the channels beneath the wells of the manifold are shown.

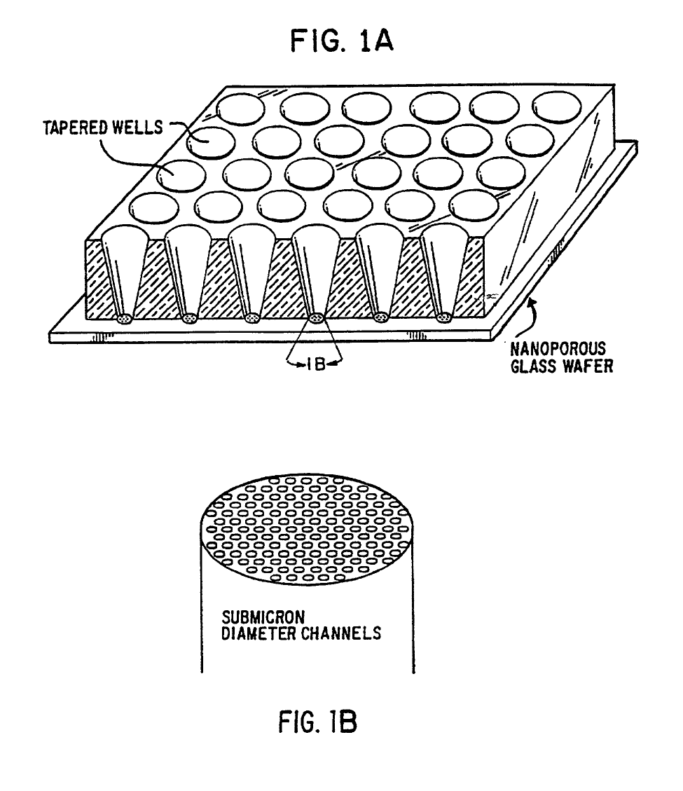

FIG. 2 depicts a wafer substrate with optional manifold in a sealed lower chamber to which a vacuum may be applied so that material applied to an upper reservoir contacts with the upper surface of the substrate and is pulled through the channels of the substrate by the vacuum. An O-ring comprises the wafer-lower chamber seal.

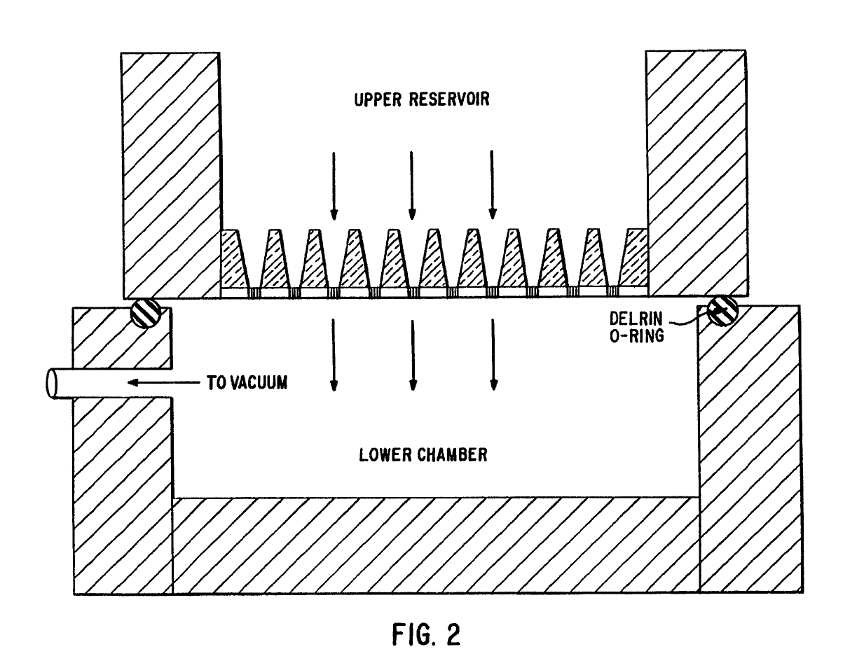

FIG. 3 depicts a silicon wafer with integral sample wells. Procedures for constructing the depicted device are described in Example 2.

FIG. 4 depicts the apparatus of FIG. 2 with a pressurized upper chamber sealed by an O-Ring.

FIGS. 5A-5E provide a schematic depiction of the results of an hprt mutation detection assay using a device in accordance with the present invention. The sequence depicted in FIG. 5B corresponds to nucleotides 23-55 of SEQ ID NO:2. One of the two sequences in FIG. 5C corresponds to nucleotides 3-22 of SEQ ID NO:4 (sequence with A in the 16th position from left) and the other to nucleotides 3-22 of SEQ ID NO:5 (bottom sequence with G replacing A at position 16).

FIG. 6 provides an idealized schematic depiction of a hybridization assay performed to profile gene expression under different experimental conditions. Details of the assay procedure are provided in Example 11.

### DETAILED DESCRIPTION

Novel flow-through devices for carrying out and detecting binding reactions are provided, in which binding reagents (or “probes”) are immobilized within channels densely packed in a solid substrate. The solid substrate contains a first and second surface, where the channels extend through the substrate from the first to the second surface. The first and second surfaces of the substrate may be planar, and also may be parallel, although non-planar and non-parallel surfaces may be used. Suitable substrate materials include microchannel or nanochannel glass and porous silicon, which may be produced using known microfabrication techniques.

Binding to reagents in the flow-through devices can be detected by devices and methods that are well known in the art including, but not limited to, microfabricated optical and electronic detection components, film, charge-coupled-device arrays, camera systems and phosphor storage technology.

Devices of the present invention overcome limitations inherent in current solid phase methods for detecting binding reactions by eliminating the diffusion-limited step in flat surface binding reactions, and by increasing the amount of binding reagent present per unit area of the two-dimensional surface on the face of the substrate.

In a particular illustrative embodiment in this regard, the device may be used as a “genosensor,” where the binding reagent is an oligonucleotide or polynucleic acid that is immobilized in the channels of the substrate, and in which the analyte is a nucleic acid that is detected by hybridization (base pairing) to the binding reagent. Particular embodiments provide some or all of the following advantages (among others) over conventional devices for detecting binding reactions:

(1) improved detection sensitivity due to the vastly increased surface area of binding reagent to which the analyte is exposed. This increased area is due to the greater surface area of the channel surfaces compared to conventional devices where the binding agent is restricted to the two-dimensional surface of the device. The presence of the binding reagent on the inner surface of the channels running through the substrate greatly increases the quantity of binding reagent present per unit of total two-dimensional substrate surface. In simple geometrical terms, for cylindrical channels of radius r extending between parallel surfaces of a substrate having a thickness h, the inner surface area is given by π2rh. By contrast, for binding reagent confined only to the two-dimensional surface of a substrate, the surface area is given by πr². Accordingly, for a single channel, the device of the invention can be considered to increase the surface area available for carrying binding reagent by a factor of 2h/r. For a channel of radius 5 micrometers in a substrate 500 micrometers thick, this results in a 200-fold increase in the surface area. In a more complex example, where a group of channels of radius r contains n channels arranged in a circle of radius R, the two-dimensional area on the surface of the substrate is defined by πR², whereas the surface area inside the channels is given by nπ2rh. Accordingly, the increase in surface area is defined by the ratio: nπ2rh/πR². Taking the above example, for instance, when r=5 and R=50 and there are 20 channels per group, this results in an increase in a 20-fold increase in the surface area.

(2) minimization of a rate-limiting diffusion step preceding the hybridization reaction (reducing the time required for the average target molecule to encounter a surface-tethered binding reagent or probe from hours to milliseconds, speeding hybridization and enabling mismatch discrimination at both forward and reverse reactions;

(3) improved analysis of dilute nucleic acid solutions by gradually flowing the solution through the channels in the wafer;

(4) facilitates recovery of bound nucleic acids from specific hybridization sites within the array, enabling further analysis of the recovered molecules;

(5) facilitates chemical bonding of probe molecules to the surface within the channels by avoiding the deleterious effect of rapid drying that occurs when small droplets of probe solution on flat surfaces are exposed to the atmosphere; and

(6) confines the binding reagent within the channels, avoiding the problem where the binding reagent must somehow be prevented from spreading on a flat surface.

Accordingly, the present invention provides an improved apparatus and methods for the simultaneous conduct of a multiplicity of binding reactions on a substrate, where the substrate is a microfabricated device having channels that run from a first to a second surface of the substrate. The channels may be subdivided and/or grouped into discrete and isolated regions defined by the presence or absence of particular binding reagents. A discrete and isolated region may comprise a single channel, or may comprise a collection of adjacent channels that defines a cognizable area on the surface of the substrate.

In one embodiment, the groups of channels in each of the discrete and isolated regions each contain an essentially homogeneous sample of a biomolecule of discrete chemical structure fixed in the channels and, accordingly, each discrete and isolated region corresponds to the location of a single binding reaction.

The substrate is contacted with a sample (hereinafter, the “test sample”) suspected of containing one or more molecular species that specifically bind to one or more of the binding reagents. Detection of the regions in which such binding has taken place then yields a pattern of binding that characterizes or otherwise identifies the molecular species present in the test sample.

The invention therefore provides novel high-density and ultra-high density microfabricated devices for the conduction and detection of binding reactions. The devices of the present invention are used to characterize or otherwise identify molecular species that bind to a particular binding reagent via essentially any mode of specific molecular binding, including known modes of binding and modes that will be discovered in the future. For example, the novel devices may be used to detect: antibody-antigen and ligand-receptor binding; nucleic acid hybridization reactions, including DNA-DNA, DNA-RNA, and RNA-RNA binding; nucleic acid-protein binding, for example in binding of transcription factors and other DNA-binding proteins; and binding reactions involving intact cells or cellular organelles. In one particular embodiment, the device may be used for DNA sequence analysis.

The apparatus of the present invention thus may be employed in a variety of analytical tasks, including nucleic acid sequence analysis by hybridization, analysis of patterns of gene expression by hybridization of cellular mRNA to an array of gene-specific probes, immunochemical analysis of protein mixtures, epitope mapping, assay of receptor-ligand interactions, and profiling of cellular populations involving binding of cell surface molecules to specific ligands or receptors immobilized within individual binding sites. Specifically, the invention is not limited to the nucleic acid analysis exemplified herein, but may equally be applied to a broad range of molecular binding reactions involving small molecules, macromolecules, particles, and cellular systems. See, for example, the uses described in PCT Published Application WO 89/10977.

The device may be used in conjunction with detection technologies that are known in the art that are capable of discriminating between regions in which binding has taken place and those in which no binding has occurred. When necessary, the detection methodology is capable of quantitating the relative extent of binding in different regions. In DNA and RNA sequence detection, autoradiography and optical detection advantageously may be used, although the skilled artisan will recognize that other detection methodologies, including methods to be developed in the future, may be used. Autoradiography may be performed, for example, using ³²P or ³⁵S labelled samples, although the skilled artisan will recognize that other radioactive isotopes also may be used.

A highly preferred method of detection is a charge-coupled-device array or CCD array. With the CCD array, a individual pixel or group of pixels within the CCD array is placed adjacent to each confined region of the substrate where detection is to be undertaken. Light attenuation, caused by the greater absorption of an illuminating light in test sites with bound molecules, is used to determine the sites where binding has taken place. Lens-based CCD cameras can also be used.

Alternatively, a detection apparatus can be constructed such that sensing of changes in AC conductance or the dissipation of a capacitor placed contiguous to each conformed region can be measured. Similarly, by forming a transmission line between two electrodes contiguous to each confined region, bound molecules can be measured by the radio-frequency (RF) loss. Methods suitable for use herein are described in, Optical and Electrical Methods and Apparatus for Molecule Detection, PCT Published Application WO 93/22678, published Nov. 11, 1993, and expressly incorporated herein by reference.

In a particular embodiment, the present invention provides improved “genosensors, ” that may be used, for example, in the identification or characterization of nucleic acid sequences through nucleic acid probe hybridization with samples containing an uncharacterized polynucleic acid, e.g., a cDNA, mRNA, recombinant DNA, polymerase chain reaction (PCR) fragments or the like, as well as other biomolecules.

Two fundamental properties of DNA are vital to its coding and replicational functions in the cell:

(1) The arrangement of “bases” [adenenine (A), guanine (G), cytosine (C) and thymine (T)] in a specific sequence along the DNA chain defines the genetic makeup of an individual. DNA sequence differences account for the differences in physical characteristics between species and between different individuals of a given species

(2) One strand of DNA can specifically pair with another DNA strand to form a double-stranded structure in which the bases are paired by specific hydrogen bonding: A pairs with T and G pairs with C. Specific pairing also occurs between DNA and another nucleic acid, ribonucleic acid (RNA), wherein uracil (U) in RNA exhibits the same base pairing properties as T in DNA.

The specific pattern of base pairing (A with T or U and G with C) is vital to the proper functioning of nucleic acids in cells, and also comprises a highly specific means for the analysis of nucleic acid sequences outside the cell. A nucleic acid strand of specific base sequence can be used as a sequence recognition element to “probe” for the presence of the perfectly “complementary” sequence within a nucleic acid sample (Conner et al., Proc. Natl. Acad. Sci., U.S.A., 80:278-282 (1983)). Thus, if a sample of DNA or RNA is “annealed” or “hybridized” with a nucleic acid “probe” containing a specific base sequence, the probe will bind to the nucleic acid “target” strand only if there is perfect (or near-perfect) sequence complementarily between probe and target. The hybridization event which indicates the presence of a specific base sequence in a nucleic acid sample may be detected by immobilization of the nucleic acid sample or the probe on a surface, followed by capture of a “tag” (for example, radioactivity or fluorescence) carried by the complementary sequence.

DNA hybridization has been employed to probe for sequence identity or difference between DNA samples, for example in the detection of mutations within specific genetic regions (Kidd et al., N. Engl. J. Med., 310:639-642 (1984); Saiki et al., N. Engl. J. Med., 319:537-541 (1988); Saiki et al., Proc. Natl. Acad. Sci. U.S.A., 86:6230-6234 (1989)). Although DNA probe analysis is a useful means for detection of mutations associated with genetic diseases, the current methods are limited by the necessity of performing a separate hybridization reaction for detection of each mutation.

Many human genetic diseases, for example, cancer (Hollstein et al., Science, 253:49-53 (1991)) are associated with one or more of a large number of mutations distributed at diverse locations within the affected genes. In these cases it has been necessary to employ laborious DNA sequencing procedures to identify disease-associated mutations. The problem is compounded when there is a need to analyze a large number of DNA samples, involving populations of individuals. Detection of mutations induced by exposure to genotoxic chemicals or radiation is of interest in toxicology testing and population screening, but again, laborious, costly and time consuming procedures are currently necessary for such mutational analyses.

In addition to causing genetic diseases, mutations also are responsible for DNA sequence polymorphisms between individual members of a population. Genetic polymorphisms are DNA sequence changes at any given genetic locus which are maintained in a significant fraction of the individuals within a population. DNA sequence polymorphisms can serve as useful markers in genetic mapping when the detectable DNA sequence changes are closely linked to phenotypic markers and occur at a frequency of at least 5% of the individuals within a population. In addition, polymorphisms are employed in forensic identification and paternity testing.

Currently employed methods for detecting genetic polymorphisms involve laborious searches for “restriction fragment length polymorphisms” (RFLPS) (Lander et al., Proc. Natl. Acad, Sci. U.S.A., 83:7353-7357 (1986)), the likewise laborious use of gel electrophoretic DNA length analysis, combined with a DNA amplification procedure which utilizes oligonucleotide primers of arbitrary sequence (Williams et al., Nucl. Acids Res., 18:6531-6535 (1991); Welsh et al., Nucl. Acids Res., 18:7213-7218 (1991)), and the gel electrophoretic analysis of short tandem repeat sequences of variable length) in genomic DNA. Weber et al., Genomics 7:524-530 (1990) and Am. J. Hum. Genet. 44:388-396 (1989).

Another kind of DNA sequence variation is that which occurs between species of organisms, which is of significance for several reasons. First, identification of sequence differences between species can assist in determination of the molecular basis of phenotypic differences between species. Second, a survey of sequence variation within a specific gene among numerous related species can elucidate a spectrum of allowable amino acid substitutions within the protein product encoded by the gene, and this information is valuable in the determination of structure-function relationships and in protein engineering programs. However, this type of targeted DNA sequence comparison is extremely laborious, time consuming and costly if carried out by current DNA sequencing methodology. Additionally, genetic sequence variation can form the basis of specific identification of organisms, for example, infectious micro-organisms.

For traditional DNA sequence analysis applications, nucleic acid fragments are end-labeled with ³²P and these end-labeled fragments are separated by size and then placed adjacent to x-ray film as needed to expose the film, a function of the amount of radioactivity adjacent to a region of film. Alternatively, phosphorimager detection methods may be used.

Optical detection of fluorescent-labeled reporters may also be employed in detection. In traditional sequencing, a DNA base-specific fluorescent dye is attached covalently to the oligonucleotide primers or to the chain-terminating dideoxynucleotides used in conjunction with DNA polymerase. The appropriate absorption wavelength for each dye is chosen and used to excite the dye. If the absorption spectra of the dyes are close to each other, a specific wavelength can be chosen to excite the entire set of dyes. One particularly useful optical detection technique involves the use of ethidium bromide, which stains duplex nucleic acids. The fluorescence of these dyes exhibits an approximate twenty-fold increase when it is bound to duplexed DNA or RNA, when compared to the fluorescence exhibited by unbound dye or dye bound to single-stranded DNA. This dye is advantageously used to detect the presence of hybridized polynucleic acids.

Methods for attaching samples of substantially homogeneous biomolecules to the channels of the microapparatus are known in the art. One preferred method of doing so is to attach such biomolecules covalently to surfaces such as glass or gold films. For example, methods for attachments of oligonucleotide probes to glass surfaces are known. A primary amine is introduced at one terminus during the chemical synthesis thereof. Optionally, one or more triethylene glycol units may be introduced therebetween as spacer units. After derivatizing the glass surface in the confined region with epoxysilane, the primary amine terminus of the oligonucleotide can be covalently attached thereto. See Beattie et al., cited above, for a further description of this technology for fixing the pre-determined biomolecules in the bounded regions of the microfabricated apparatus.

The present invention, thus generally described, will be understood more readily by reference to the following examples, which are provided by way of illustration and are not intended to be limiting of the present invention.

### EXAMPLE 1

### Nanochannel Glass (NCG) Wafers

Nanochannel glass arrays developed at the Naval Research Laboratory can be used in the present invention to provide a high surface area nanochannel substrate to tether binding reagents such as DNA targets or probes for hybridization. NCG materials are glass structures containing a regular geometric array of parallel holes or channels as small as 33 nm in diameter or as large as a hundred micrometers or more in diameter. See Tonucci et al., Science 258:783-785 (1992), and U.S. Pat. No. 5,234,594 which are incorporated herein by reference in their entireties. These nanochannel glass structures can be fabricated in various array configurations to provide a high surface area to volume ratio, and can possess packing densities in excess of 3×10¹⁰ channels per square centimeter. A variety of materials can be immobilized or fixed to the glass surfaces within the channels of the NCG array.

Nanochannel glass arrays are fabricated by arranging dissimilar glasses in a predetermined configuration where, preferably, at least one glass type is usually acid etchable. Typically, a two-dimensional hexagonal close packing array is assembled from etchable glass rods (referred to as the channel glass) and an inert glass tube (referred to as the matrix glass). The pair is then drawn under vacuum to reduce the overall cross-section to that of a fine filament. The filaments are then stacked, re-fused and redrawn. This process is continued until appropriate channel diameters and the desired number of array elements are achieved. By adjusting the ratio of the diameter of the etchable glass rod to that of the outside dimension of the inert glass tubing, the center-to-center spacing of the rods and their diameters in the finished product become independently adjustable parameters. See Tonucci, supra.

Once the fabrication process is complete, the NCG material is wafered perpendicular to the direction of the channels with a diamond saw and then polished to produce sections of material having a defined thickness, for example, about 0. 1 mm to about 1.0 mm. The channel glass of the array structure is then etched away with an acid solution. The skilled artisan will recognize that other geometries of the substrate are possible. For example, the opposing faces of the substrate need not be parallel, and the substrate may be thinner or thicker than about 0.1 mm to about 1.0 mm. For example, the thickness of the substrate can range from about 10 μm to about 250 μm, from about 50 to about 500 μm, from about 250 μm to about 1.5 mm, or about 500 μm to about 2 mm thick. Moreover, the skilled artisan will appreciate that the cross-sectional configuration of the channels may be varied. For example, the geometry of the channels may include, but is not limited to, a circular or hexagonal cross-section.

In one particular example, a hexagonal close packing arrangement of channel glasses is used which, after acid etching, contains typically 10⁷ channels that are uniformly dispersed in the substrate. The channel diameter is typically 450 nm and the center-to-center spacing is approximately 750 nm. The skilled artisan will recognize, however, that the channel diameter can be wider or narrower than 450 nm, and the center-to-center spacing also may be varied. Variation in the channel geometry allows for design of variation in the density of the channels in the substrate. The type of array structure described above is useful in the NCG array assembly in accordance with the present invention. As noted above, a manifold containing sample wells can be used to define group of channels that each serve as sites for specific binding reactions. As described infra, however, other methods of defining groups of channels also may be used.

A second example of hexagonal array structure is one in which separated clusters of channels are formed during the fabrication process. For example, an open array structure with typical channel diameters of 300 nm in which the overall glass structure consists of an array of 18 μm diameter subarrays, spaced typically 25 μm apart from neighboring arrays. Once again, the skilled artisan will recognize that the diameters of the channels and the subarrays and their spacing can be varied without departing from the spirit of the invention.

### EXAMPLE 2

### Silicon Wafers

Two illustrative general types of silicon devices containing channels between a first and second surface of the device that can be prepared according to the process are described herein below.

Silicon designs containing channels are advantageously employed because of their adaptability to low cost mass production processes and their ability to incorporate in the fabrication process structural elements that function in fluidic entry and exit from the hybridization site and structures (e.g., electrodes) that may function in hybridization detection. Stable, open-cell materials containing channels between first and second surfaces of the material are used to accomplish enhancements and to introduce qualitatively new features in these devices, whereby the surface area of discrete and isolated binding regions comprising groups of channels is increased by a factor of 100 to 1000 relative to a two-dimensional surface.

Thin-film processing technology is used to deposit chemically inert and thermally stable microchannel materials. Materials and processing methods are selected to achieve low-cost semiconductor batch fabrication of integrated semiconductor detectors. The microchip device provides in situ multisite analysis of binding strength as ambient conditions are varied. Silicon materials containing channels are fabricated in oriented arrays with channel diameters selected over the range from 2 nm to several micrometers. Random, interconnected pore arrays also can be made.

Porous silicon is produced most easily through electrochemical etching. It can be processed into two important channel structures, interconnected networks and oriented arrays. The channel diameter is tailored from approximately 2 nm to micrometer dimensions by selection of doping and electrochemical conditions. For n-type material, etching is thought to proceed through a tunneling mechanism in which electrons are injected into the channel surface through field concentration effects. In the case of p-material the mechanism seems to be through moderation of carrier supply at the electrolyte/silicon interface. In practice, the following structures can be fabricated for use as suitable substrates for the present invention:

i) dense oriented arrays of channels oriented with axis along <100>direction and with channel diameters in the range of 10 to 100 nm. Obtained in p-type material with resistivity less than 10-2 Ω-cm.

ii) dense oriented arrays of channels oriented along <100> direction and with channel diameters in the range less than 10 nm. Obtained in n-type material with resistivity between 10-1 and 10-2 Ω-cm.

iii) dense oriented arrays of rectangular channels oriented with axis along <100> direction, rectangle side defined by {001 } planes, and with channel diameters in range less than 100 nm. Obtained in p-type material with resistivity between 10-1 and 10-2 Ω-cm.

Characterization can be undertaken by scanning electron microscopy. The surface wetting properties are varied using vapor treatment with silylation materials and chlorocarbons.

High channel-density dielectrics which function as molecular sieves are produced by nuclear track etching. While nuclear track etching is used to produce these molecular sieves in a wide range of inorganic materials, it is most often used with dielectrics such as mica and sapphire. In this method, described in U.S. Pat. No. 3,303,085 (Price, et al., which is hereby incorporated by reference in its entirety), a substrate is first bombarded with nuclear particles (typically several MeV alpha particles) to produce disturbances or “tracks” within the normal lattice structure of the material and then wet-etched to produce channels which follow the tracks caused by the nuclear particles. More specifically, Price et al. disclose that the exposure of a mica substrate to heavy, energetic charged particles will result in the formation of a plurality of substantially straight tracks in its lattice structure and that these tracks can be converted into channels by wet etching the substrate.

Channel sizes and density of the channels are variably controllable with channels typically 0.2 μm in diameter and densities on the order of 10⁹/cm², although narrower or broader channels can be generated, leading to greater or smaller channel densities. Particle track depths are energy dependent on the incident particle beam, but resulting channels can be extended, for example, through an entire 500 μm-thick substrate. Incorporation of these materials on the device shown above is readily accomplished. In addition, the use of implantation-etched dielectrics as the sensor element has advantages versus the silicon approach since the material is hydrophilic.

Known microfabrication methods can be used to fabricate manifold structures defining, for instance, integral sample wells that can be used to direct binding reagents or samples towards specific locations on the binding device. A binding device formed from a wafer structure having uniform channels can be bonded to the manifold as described below (see Example 3) for NCG glass arrays.

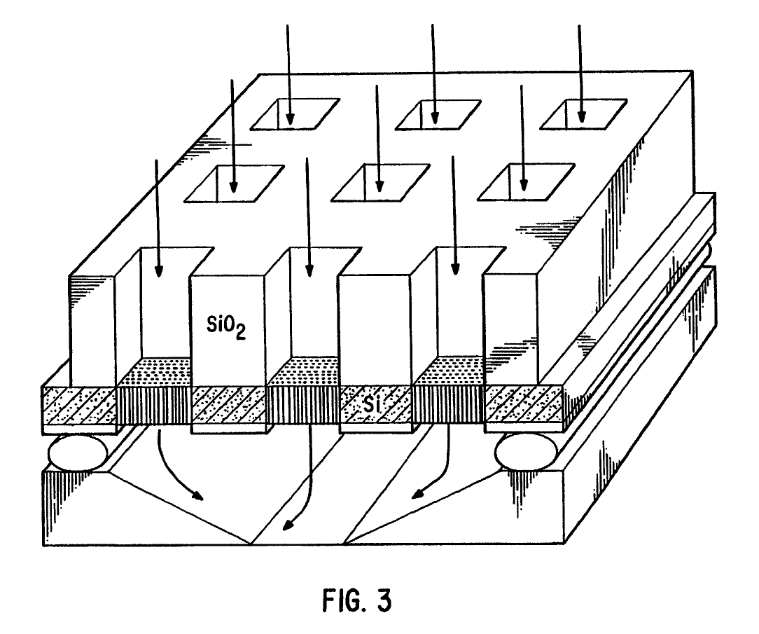

A preferred device in this regard is the silicon array wafer containing channels between first and second surfaces of the wafer, and containing integral sample wells as illustrated in FIG. 3. By way of example, this may be constructed as follows: A four inch diameter, 100 μm thick wafer of crystalline silicon (n-type, doped with 1015 P/cm³) with axis oriented along <100> direction is coated with photoresist and exposed to light through a mask to define a 50×50 array of 200 μm square areas having 200 μm space between them across the 2 cm×2 cm central area of the wafer. The process described by V. Lehmann (J Electrochem. Soc. 140:2836-2843 (1993)) is then used to create patches of closely spaced channels of diameter 2-5 μm oriented perpendicular to the wafer surface, within each square area defined in the photolithographic step. A 300 μm thick wafer of silicon dioxide is coated with photoresist and exposed to light through the same mask used to define 200 μm square channel regions in the silicon wafer, and acid etching is conducted to create 200 μm square holes in the silicon dioxide wafer. The silicon dioxide wafer is then aligned with and laminated to the silicon wafer using a standard wafer bonding process to form the integral structure shown in the figure. During the high temperature annealing step, the silicon surface of each channel is oxidized to form a layer of silicon dioxide.

The size of the silicon array wafers may be modified in a variety of ways without departing from the spirit of the invention.

### EXAMPLE 3

### Well Arrays Defining Discrete and Isolated Binding Regions Manifold)

The NCG hybridization arrays described in Example 1 can be bonded to an array of orifices which align with the array of channels and serve as wells for placement of binding molecules, for instance, a substantially homogeneous sample of a biomolecule (e.g., a single DNA species) in defined sites (groups of channels) on the substrate. Such well arrays also can provide physical support and rigidity to the substrate such as a NCG wafer.

Polymeric well arrays can be fabricated using methods known in the art. For example, a polymeric layer suitable for use herein can be obtained from MicroFab Technologies, Inc., and the orifices can be fabricated using excimer laser machining. This method is preferred because existing technology is employed, allowing for low cost/high volume manufacturing.

Development of the polymeric array comprises: (1) materials selection; (2) ablation tooling and process development; (3) lamination tooling and process development; and (4) production of high density and ultra-high density polymeric arrays. These tasks are undertaken as follows:

Part A: Materials Selection

The materials useful in the polymeric array are filled polymers, epoxy resins and related composite (e.g., “circuit-board”-type) materials. Because it is a standard process in the microelectronics industry, the present invention most advantageously employs polymeric materials with the adhesive applied by the commercial vendor of the material, for example, a polyamide with a 12 μm thick layer of a B-stage (heat curing) adhesive.

The primary requirements for the polymeric array material to be used are:

1. High suitability for excimer laser machinability:

i. high absorption in UV (e.g., >4×10⁵/cm at 193 nm);

ii. high laser etch rate (e.g., 0.5 μm/pulse) and low hole taper (reduction in hole diameter with depth into material, e.g., <3°);

2. Obtainable in thicknesses up to 1 mm;

3. Obtainable with B-stage adhesive on one side which is both laser ablatable and suitable for bonding to the nanochannel wafer;

4. High rigidity and thermal stability (to maintain accurate alignment of samplewell and NCG wafer features during lamination);

5. Compatibility with DNA solutions (i.e., low nonspecific binding)

Part B: Ablation Tooling and Process

Contact mask excimer laser machining is a preferred processing technique use because it is a lower cost technique than projection mask excimer laser machining. A projection mask is, however, employed when the feature size is less than 50 μm. One or more masks with a variety of pattern sizes and shapes are fabricated, along with fixtures to hold the mask and material to be ablated. These masks are employed to determine the optimal material for laser machining and the optimal machining conditions (i.e., mask hole size, energy density, input rate, etc.). Scanning electron microscopy and optical microscopy are used to inspect the excimer laser machined parts, and to quantify the dimensions obtained, including the variation in the dimensions.

In addition to ablating the sample wells into the polymeric material, the adhesive material also is ablated. This second ablation is undertaken so that the diameter of the hole in the adhesive is made larger than the diameter of the sample well on the adhesive side of the polymeric material. This prevents the adhesive from spreading into the sample well and/or the nanochannel glass during lamination.

Part C: Lamination Tooling and Processing

Initial lamination process development is carried out using unablated polymeric material (or alternatively, using glass slides and/or silicon wafers). Cure temperature, pressure, and fixturing are optimized during this process development. Thereafter, the optimized processing parameters are employed to laminate both nanochannel wafers and polymeric arrays. The final lamination is done such that the alignment of the two layers creates functional wells.

Part D: Production of Polymeric Arrays

The optimal mask patterns and excimer laser parameters are determined and thereafter employed in the manufacture of contact masks and material holding fixtures. Typically, fabrication is done so as to produce a large number (>100) of parts as the masks wear out with use.

### EXAMPLE 4

### Robotic Fluid Delivery

Delivery of binding reagent to defined locations within a microchannel substrate is accomplished in certain embodiments using micro-spotting devices, as illustrated below.

A. Hamilton Microlab 2000

A Hamilton Microlab 2200 robotic fluid delivery system, equipped with special low volume syringes and 8-position fluid heads, capable of delivering volumes of 10-100 nl at 500 μm xyz stepping and a few percent precision. Using this equipment 40-nl samples of biomolecules (e.g., DNA, oligonucleotides and the like) are placed into the wells of the high density NCG wafer. A piezoelectrically controlled substage custom fitted for the Microlab 2200 permits xy positioning down to submicron resolution. Custom fabricated needles are employed. The eight-needle linear fluid head is operated in staggered repetitive steps to generate the desired close spacing across the wafer. The system has a large stage area and rapid motion control, providing capacity to produce hundreds of replicate hybridization wafers.

B: Microfab Microfluidic Jets

Methods are known in the art and devices are commercially available (Microfab Technologies, Inc.) for delivering microdroplets of fluids to a surface with great precision. A microjet system capable of delivering subnanoliter DNA solutions to the wafer surface is employed as follows: For placement of DNA into individual hybridization sites within ultra-high density wafers, with volumes of one nl (corresponding to a 130 μm sphere or 100 μm sphere or 100 μm cube) commercially available dispensing equipment using ink-jet technology as the microdispensing method for fluid volume below is employed.

The droplets produced using ink-jet technology are highly reproducible and can be controlled so that a droplet may be placed on a specific location at a specific time according to digitally stored image data. Typical droplet diameters for demand mode ink-jet devices are 30-100 μm, which translates to droplet volumes of 14-520 pl. Droplet creation rates for demand mode ink-jet devices are typically 2,000-5,000 droplets per second. Thus, both the resolution and throughput of demand mode inkjet microdispensing are in the ranges required for the ultrahigh density hybridization wafer.

C: Microdispensing System

The microdispensing system is modified from a MicroFab drop-on-demand inkjet type device, hereafter called a MicroJet device such that this type of device can produce 50 μm diameter droplets at a rate of 2000 per second. The operating principles of this type of device are known (Wallace, “A Method of Characteristics Model of a Drop-On-Demand Ink-Jet Device Using an Integral Drop Formation Method, ” ASME publication 89-WA/FE-4, December 1989) and used to effect the modification. To increase throughput, eight of these devices are integrated into a line array less than 1 inch (25 mm) long. The eight devices are loaded with reagent simultaneously, dispense sequentially, and flush simultaneously. This protocol is repeated until all of the reagents are dispensed. Most of the cycle time is associated with loading and flushing reagents, limiting the advantages of a complex of parallel dispensing capability. Typical cycle time required is as on the following order: 1 minute for flush and load of 8 reagents; 30 seconds to calibrate the landing location of each reagent; 15 seconds to dispense each reagent on one location of each of the 16 genosensors, or 2 minutes to dispense all 8 reagents. Total time to load and dispense 8 reagents onto 16 sensors is thus 3.5 minutes. Total time for 64 reagents onto 16 sensors would be 28 minutes. The microdispensing system will consist of the subsystems listed below:

1. Microjet Dispense Head

An assembly of 8 MicroJet devices and the required drive electronics. The system cost and complexity are minimized by using a single channel of drive electronics to multiplex the 8 dispensing devices. Drive waveform requirements for each individual device are downloaded from the system controller. The drive electronics are constructed using conventional methods.

2. Fluid Delivery System

A Beckman Biomec is modified to act as the multiple reagent input system. Between it and the MicroJet dispense head are a system of solenoid valves, controlled by the system controller. They provide pressurized flushing fluid (deionized water or saline) and air to purge reagent from the system and vacuum to load reagent into the system.

3. X-Y Positioning System

A commercially available precision X-Y positioning system, with controller, is used. Resolution of 0.2 μm and accuracy of 2 μm are readily obtainable. The positioning system is sized to accommodate 16 sensors, but MicroJet dispense head size, purge station, and the calibration station represent the main factors in determining overall size requirements.

4. Vision System

A vision system is used to calibrate the “landing zone” of each MicroJet device relative to the positioning system. Calibration occurs after each reagent loading cycle. Also, the vision system locates each dispensing site on each sensor when the 16 sensor tray is first loaded via fiducial marks on the sensors. For economy, a software based system is used, although a hardware based vision system can be advantageously employed.

5. System Controller

A standard PC is used as the overall system controller. The vision system image capture and processing also reside on the system controller.

### EXAMPLE 5

### Oligonucleotide Attachment to Glass/SiO2

Part A: Epoxysilane Treatment of Glass

A stock solution of epoxysilane is freshly prepared with the following proportions: 4 ml 3-glycidoxypropyl-trimethoxysilane, 12 ml xylene, 0.5 ml N,N-diisopropylethylamine (Hünig's base). This solution is flowed into the channels of the wafer, followed by soaking for 5 hours in the solution at 80° C., followed by flushing with tetrahydrofuran, drying at 80° C., and drying in a vacuum desiccator over Drierite or in a desiccator under dry argon.

Part B: Attachment of Oligonucleotide

Oligonucleotide, bearing 5′- or 3′-alkylamine (introduced during the chemical synthesis) is dissolved at 10 mM-50 mM in water and flowed into the channels of the silica wafer. After reaction at 65° C. overnight the surface is briefly flushed with water at 65° C., then with 10 mM triethylamine to cap off the unreacted epoxy groups on the surface, then flushed again with water at 65° C. and air dried. As an alternative to attachment in water, amine-derivatized oligonucleotides can be attached to epoxysilane-derivatized glass in dilute (eg., 10 mM-50 mM) KOH at 37° C. for several hours, although a higher background of nonspecific binding of target sample DNA to the surface (independent of base pairing) may occur during hybridization reaction.

### EXAMPLE 6

### Liquid Flow-through

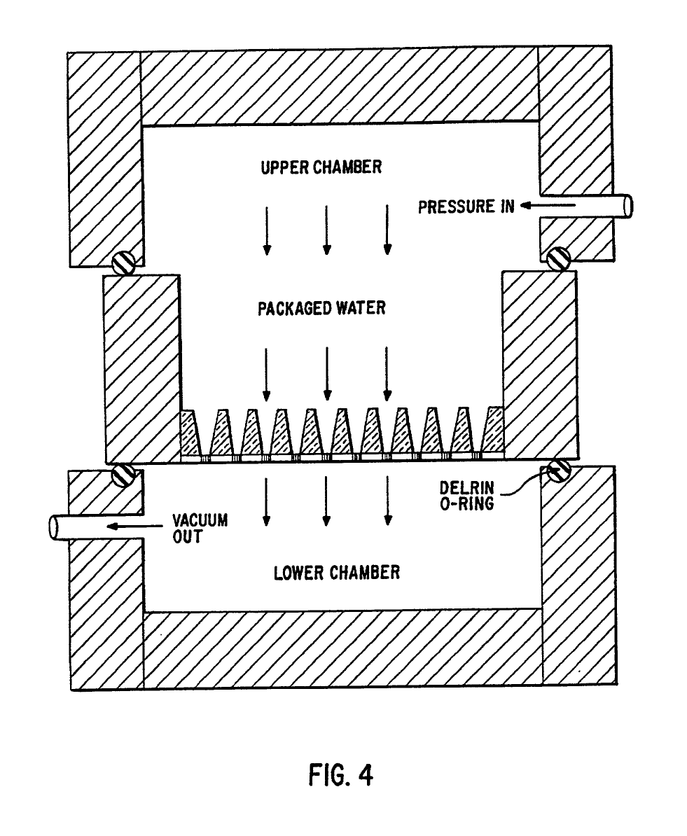

In order to bind DNA probes or targets within the channels of the microfabricated hybridization support, carry out the hybridization and washing steps, process the material for re-use, and potentially recover bound materials for further analysis, a method of flowing the liquids through the wafer is provided. To enable flow of liquid through the hybridization wafer, the wafer is packaged within a 2 mm×4 mm polypropylene frame, which serves as an upper reservoir and structure for handling. A polypropylene vacuum chamber with a Delrin o-ring around its upper edge permits clamping of the wafer onto the vacuum manifold to form a seal. The vacuum assembly is illustrated in FIG. 4. For control of fluid flow through the wafer a screw-drive device with feedback control is provided.

### EXAMPLE 7

### Synthesis and Derivatization of Oligonucleotides

Oligonucleotides to be used in the present invention are synthesized by phosphoramidite chemistry (Beaucage et al. Tet. Lett. 22:1859-1862 (1981)) using an segmented synthesis strategy that is capable of producing over a hundred oligonucleotides simultaneously (Beattie et al., Biotechnol. Appl. Biochem. 10:510-521 (1988); Beattie et al., Nature 352:548-549 (1991)). The oligonucleotides can be derivatized with the alkylamino function during the chemical synthesis, either at the 5′-end or the 3′-end.

Part A: Chemistry of Attachment to Glass

Optimal procedures for attachment of DNA to silicon dioxide surfaces are based on well-established silicon chemistry (Parkam et al., Biochem. Biophys. Res. Commun., 1:1-6 (1978); Lund et al., Nucl. Acids Res. 16:10861-10880, (1988)). This chemistry is used to introduce a linker group onto the glass which bears a terminal epoxide moiety that specifically reacts with a terminal primary amine group on the oligonucleotide. This versatile approach (using epoxy silane) is inexpensive and provides a dense array of monolayers that can be readily coupled to terminally modified (amino- or thiol-derivatized) oligonucleotides. The density of probe attachment is controlled over a wide range by mixing long chain amino alcohols with the amine-derivatized oligonucleotides during attachment to epoxysilanized glass. This strategy essentially produces a monolayer of tethered DNA, interspersed with shorter chain alcohols, resulting in attachment of oligonucleotides down to 50 apart on the surface. Variable length spacers are optionally introduced onto the ends of the oligonucleotides, by incorporation of triethylene glycol phosphoryl units during the chemical synthesis. These variable linker arms are useful for determining how far from the surface oligonucleotide probes should be separated to be readily accessible for pairing with the target DNA strands. Thiol chemistry, adapted from the method of Whitesides and coworkers on the generation of monolayers on gold surfaces (Lee et al. Pure & Appl. Chem. 63:821-828 (1991) and references cited therein.), is used for attachment of DNA to gold and platinum surfaces. Dithiols (e.g., 1,10-decanedithiol) provide a terminal, reactive thiol moiety for reaction with bromoacetylated oligonucleotides. The density of attachment of DNA to gold or platinium surfaces is controlled at the surface-activation stage, by use of defined mixtures of mono- and dithiols.

Part B: Surface Immobilization of Recombinant Vector DNA, cDNA and PCR Fragments

The chemical procedures described above are used most advantageously for covalent attachment of synthetic oligonucleotides to surfaces. For attachment of longer chain nucleic acid strands to epoxysilanized glass surfaces, the relatively slow reaction of surface epoxy groups with ring nitrogens exocylic amino groups along the long DNA strands is employed to achieve immobilization. Through routine experimentation, optimal conditions for immobilization of unmodified nucleic acid molecules at a few sites per target are defined, such that the bulk of the immobilized sequence remains available for hybridization. In the case of immobilization to nanochannels coated with platinum or gold, hexylamine groups are first incorporated into the target DNA using polymerization (PCR or random priming) in the presence of 5-hexylamine-dUTP, then a bromoacetylation step is carried out to activate the DNA for attachment to thiolated metal surfaces. Again, routine experimentation is employed (varying the dTTP/5-hexylamine-dUTP ratio and the attachment time) to define conditions that give reproducible hybridization results.

The foregoing procedure (omitting the bromoacetylation step) can also serve as an alternative method for immobilization of target DNA to glass surfaces.

Part C: DNA Binding Capacity

Based upon quantitative measurements of the attachment of labeled oligonucleotides to flat glass and gold surfaces, the end attachment places the probes 50-100 nm apart on the surface, corresponding to up to 10⁸ probes in a 50 μm×50 μm area. Approximately 10¹⁰-10¹¹ oligonucleotide probes can be tethered within a 50 μm cube of silicon in the nanofabricated wafer. The density of bound oligonucleotides per cross sectional area is estimated by end-labeling prior to the attachment reaction, then quantitating the radioactivity using the phosphorimager. Known quantities of labeled oligonucleotides dried onto the surface are used to calibrate the measurements of binding density. From data on the covalent binding of hexylamine-bearing plasmid DNA to epoxysilanized flat glass surfaces in mild base, it is known that at least 10⁷ pBR322 molecules can be attached per mm² of glass surface. Based on this density within the channels of the nanofabricated wafer, immobilization of 10⁹-10¹⁰ molecules of denatured plasmid DNA per mM² of wafer cross section are achieved.

### EXAMPLE 8

### Hybridization Conditions

Part A: Sample Preparation

The target DNA (analyte) is prepared by the polymerase chain reaction, incorporating [³²P]nucleotides into the product during the amplification or by using gamma-³²P[ATP]+polynucleotide kinase to 5′-label the amplification product. Unincorporated label is removed by Centricon filtration. Preferably, one of the PCR fragments is 5′-biotin-labeled to enable preparation of single strands by streptavidin affinity chromatography. The target DNA is dissolved in hybridization buffer (50 mM Tris-HCl, pH 8, 2 mM EDTA, 3.3M tetramethylammonium chloride) at a concentration of at least 5 nM (5 fmol/μl) and specific activity of at least 5,000 cpm/fmol. PCR fragments of a few hundred bases in length are suitable for hybridization with surface-tethered oligonucleotides of at least octamer length.

Part B: Hybridization.

The target DNA sample is flowed into the channels of the chip and incubated at 6° C. for 5-15 minutes, then washed by flowing hybridization solution through the chip at 18° C. for a similar time. Alternatively, hybridization can be carried out in buffer containing 1M KCl or NaCl or 5.2M Betaine, in place of tetramethylammonium chloride.

Part C: Optimization of Hybridization Selectivity (Discrimination Against Mismatch-containing Hybrids)

Although the experimental conditions described above generally yield acceptable discrimination between perfect hybrids and mismatch-containing hybrids, some optimization of conditions may be desirable for certain analyses. For example, the temperature of hybridization and washing can be varied over the range 5° C. to 30° C. for hybridization with short oligonucleotides. Higher temperatures may be desired for hybridization using longer probes.

### EXAMPLE 9

### Quantitative Detection of Hybridization

Part A: Phosphorimager and Film Detection

The detection and quantitation of hybridization intensities is carried out using methods that are widely available: phosphorimager and film. The Biorad phosphorimager has a sample resolution of about 100 μm and is capable of registering both beta emission and light emission from chemiluminescent tags. Reagent kits for chemiluminescence detection available from Millipore and New England Nuclear, which produce light of 477 and 428 nm, respectively, are advantageously used with the Biorad instrument. Chemiluminescent tags are introduced into the target DNA samples (random-primed vector DNA or PCR fragments) using the procedures recommended by the supplier. Thereafter, the DNA is hybridized to the nanochannel wafers bearing oligonucleotide probes. Radioactive tags (³²P and ³³P, incorporated by random priming and PCR reaction) are also used in these experiments. Film exposure is used for comparison. In the case of hybridization of labeled oligonucleotides with surface immobilized target DNAs, most preferably the radioactive tags (incorporated using polynucleotide kinase) are used.

Part B: CCD Detection Devices

CCD genosensor devices are capable of maximum resolution and sensitivity and are used with chemiluminescent, fluorescent and radioactive tags (Lamture et al. supra.

### EXAMPLE 10

### Genosensor Experiment; Mutation Detection in Exon 7/8 Region of Hamster hprt Gene

The hprt gene is used extensively as a model system for studies of mutation. The gene has been cloned and sequenced from several mammals. A variety of mutations in this gene are known and were characterized by DNA sequencing, in the hamster (induced by chemicals and radiation in Chinese Hamster Ovary cell lines) and from humans (associated with Lesch Nyhan syndrome). A significant fraction of hprt mutations are found in a short region of the gene encoded by exons 7 and 8. The nucleotide sequence of the normal and mutant genes are found in the following references: Edwards et al., Genomics, 6:593-608 (1990); Gibbs et al., Genomics, 7:235-244 (1990); Yu et al., Environ. Mol. Mutagen., 19:267-273 (1992); and Xu et al., Mutat. Res., 282:237-248 (1993). The nucleotide sequence of cDNA of hamster hprt exon 7/8 region is listed as follows:

| | |
|---|---|
| GCAAGCTTGC TGGTGAAAAG GACCTCTCGA AGTGTTGGAT | (SEQ ID NO:1) |
| ATAGGCCAGA CTTTGTTGGA TTTGAAATTC CAGACAAGTT | |
| TGTTGTTGGA TATGCCCTTG ACTATAATGA GTACTTCAGG | |
| GATTTGAATC | |

The following represents the nucleotide sequence of hamster hprt genomic DNA in the exon 7/8 region where the CHO mutations are depicted above and the human (h) and mouse (m) sequence differences below. The DNA sequence which begins with “5′-aacagCTTG” and which ends with “5′-GACTgtaag” is designated as SEQ ID NO:2 for sequences of hamster, human and mouse and SEQ ID NO:3 for the sequence of CHO cells. The remaining DNA, beginning with “5′-tacagTTGT” and ending with “GAATgtaat” is designated as SEQ ID NO:4 for sequences of hamster, human and mouse and SEQ ID NO:5 the sequence of CHO cells.

```
                                                        ----------
                                                                ↑
-aacagCTTGCTGGTGAAAAGGACCTCTCGAAGTGTTGGATATAGGCCAG
                                                ↓ ↓ ↓ ↓
                                                C A C A
                                                h h m h
                                                        G −
                                                        ↑ ↑
ACTgtaag----tacagTTGTTGGATTTGAAATTCCAGACAAGTTTGTTG
                                    +A C
                                      +A C
                                        ↑ ↑
TTGGATATGCCCTTGACTATAATGAGTACTTCAGGGATTTGAATgtaat-
  ↓ ↓ ↓
  A A A
  H h h
```

The small letters in the beginning of the sequence represent intron sequence on the 5′-side of exon 7. Some flanking intron sequence between exons 7 and 8 is shown (in small letters) on the second line, and at the end there is again a small stretch of intron sequence following exon 8. Underlined bases in the sequence represent mutations for which DNA samples are available, which can be used to demonstrate that a DNA chip targeted to this region can detect and identify mutations. Above the sequences are displayed mutations in hamster (CHO) cells induced by chemicals and radiation, including a 10-base deletion (top line), single base deletion (second line), single base insertion (third line) and single base substitutions (second and third lines). Below the sequences are shown single base differences between hamster and human (h) and mouse (m).

The set of oligonucleotide probes (of 8 mer-10 mer in length) overlapping by two bases across the exon 7/8 region is depicted below for SEQ ID Nos:2-5:

```
                    ----2---- ----4---- --6--
    ----1---- ----3---- ----5---- --7--
-aacagCTTGCTGGTGAAAAGGACCTCTCGAAGTGTTGGATATAGGCCAG
                                                ↓ ↓ ↓ ↓ ↓
                                                C A −10 C A
                            ----8---- ----10---- -12-
-7- ----9---- ----11----
ACTgtaag----tacagTTGTTGGATTTGAAATTCCAGACAAGTTTGTTG
                                                        ↓ ↓
                                                        G −
--12- ----14---- ----16---- ----18----
      ----13---- ----15--- ----17----
TTGGATATGCCCTTGACTATAATGAGTACTTCAGGGATTTGAATgtaat-
  ↓ ↓ ↓ ↓
  A +A A A
                                                                    C
```

This set of probes is selected to detect any of the mutations in this region, and the lengths are adjusted to compensate for base composition effects on duplex stability (longer probes for AT-rich regions). The sequences of probes and primers are given in Table 1, as follows:

TABLE I
OLIGONUCLEOTIDES FOR hprt MUTATION DETECTION

| | |
|---|---|
| Name | Sequence (5′→3′) |

PCR primers for exons 7 & 8:

| | | |
|---|---|---|
| MHEX71 | GTTCTATTGTCTTTCCCATATGTC | (SEQ ID NO:6) |
| MHEX82 | TCAGTCTGGTCAAATGACGAGGTGC | (SEQ ID NO:7) |
| HEX81 | CTGTGATTCTTTACAGTTGTTGGA | (SEQ ID NO:8) |
| HEX82 | CATTAATTACATTCAAATCCCTGAAG | (SEQ ID NO:9) |

9mer with amine at 5′-end:

| | | |
|---|---|---|
| −A (554) | TGCTGGAAT | |
| −A (586/7) | ACTCATTTATA | (SEQ ID NO:10) |
| −10(509- | TATATGAGAG | (SEQ ID NO:11) |
| 518) | | |
| AG (545) | ATTCCAAATC | (SEQ ID NO:12) |
| GC (601) | CAAATGCCT | |
| 1 | AGCAAGCTG | |
| 2 | TTTCACCAG | |
| 3 | AGGTCCTTT | |
| 4 | CTTCGAGAG | |
| 5 | TCCAACACT | |
| 6 | GCCTATATC | |
| 7 | AGTCTGGC | |
| 8 | TCCAACAACT | (SEQ ID NO:13) |
| 9 | ATTTCAAATC | (SEQ ID NO:14) |
| 10 | GTCTGGAAT | |
| 11 | ACAAACTTGT | (SEQ ID NO:15) |
| 12 | TCCAACAAC | |
| 13 | GGGCATATC | |
| 14 | TAGTCAAGG | |
| 15 | ACTCATTATA | (SEQ ID NO:16) |
| 16 | CTGAAGTAC | |
| 17 | CAAATCCCT | |
| 18 | AATTACATFCA | (SEQ ID NO:17) |

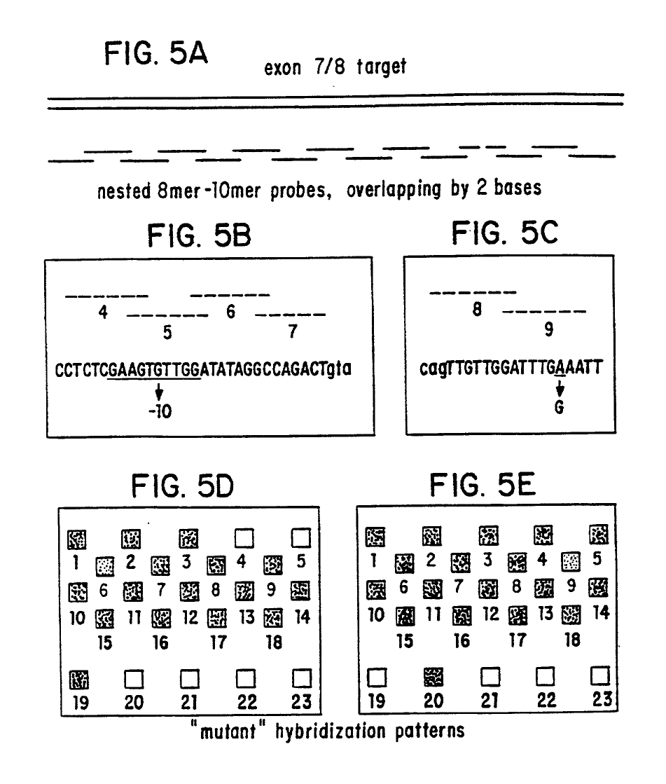

A high-density or ultra-high density microfabricated device according to the above examples is constructed and attachment of oligonucleotide probes is carried out within the bounded regions of the wafer. Included are the normal probes (1-18) plus the specific probes that correspond to five different known mutations, including the above mutations (sites 19 and 20, respectively). The foregoing uses two sets of PCR primers (Table I) to amplify the exons 7/8 region of hamster genomic DNA. A radioactive label (³²P) is incorporated into the PCR fragments during amplification, which enables detection of hybridization by autoradiography or phosphorimager. FIG. 5 illustrates the results when the above probes are attached at one end to the surface at specific test sites within the DNA chip (numbered as above). Idealized hybridization patterns for two of the mutants (10-base deletion on left and A-G transition on right) are shown at the bottom.

### EXAMPLE 11

### Profiling of Gene Expression Using cDNA Clones Arrayed in Channels in Silicon Wafers

Part A: Fabrication of Porous Silicon Wafer

The procedure outlined in EXAMPLE 3 for fabrication of a porous silicon wafer with integral wells is followed, to yield a wafer with a 50×50 array of 200 μm square patches of channels, spaced 400 μm apart (center-to-center) over the surface of the wafer. The channels of the wafer are activated to bind amine-derivatized polynucleotides by reaction with epoxysilane, as described in EXAMPLE 4.

Part B: Formation of cDNA Array

A set of 2,500 M13 clones, selected from a normalized human cDNA library, is subjected to the polymerase chain reaction (PCR) in the presence of 5′-hexylamine-dUTP to amplify the cDNA inserts and incorporate primary amines into the strands. The PCR products are ethanol-precipitated, dissolved in water or 10 mM KOH, heat-denatured at 100° C. for 5 min., then quenched on ice and applied to individual sample wells of the wafer using a Hamilton Microlab 2200 fluid delivery system equipped with an 8-needle dispensing head. After all cDNA fragments are dispensed, a slight vacuum is briefly applied from below to ensure that fluid has occupied the channels. Following incubation at room temperature overnight or at 60° C. for 30-60 minutes, the wafer is flushed with warm water, then reacted with 50 mM triethylamine to cap off the unreacted epoxy groups on the surface, then flushed again with warm water and air dried.

Part C: Preparation of Labeled PCR Fragments Representing the 3′-regions of Expressed Genes

Cytoplasmic RNA is extracted from cultured cells by the method of Chomczynski et al., (Anal. Biochem. 162:156-159 (1993)), treated with DNAse I to remove DNA contamination, then extracted with phenol/chloroform and ethanol precipitated. Reverse transcriptions and PCR are performed as described in the “differential display” protocol of Nishio et al., (FASEB J., 8:103-106 (1994)). Prior to hybridization, PCR products are labeled by random priming in the presence of [A-³²P]dNTPs, and unincorporated label is removed by Centricon filtration.

Part D: Hybridization of Expressed Sequences to cDNA Array

Prior to hybridization, a solution of 1% “Blotto” or 50 mM tripolyphosphate is flowed through the channels of the wafer to minimize the nonspecific binding of target DNA, then the porous silicon array is washed with hybridization solution (50 mM Tris-HCl, pH 7.5, 1 mM EDTA, 1M NaCl). Labeled PCR fragments representing the 3′-end of expressed genes are recovered from the Centricon filtration units in hybridization buffer, and the entire wafer is flooded with this DNA solution. The hybridization module is placed at 65° C. and a peristaltic pump, connected to the lower vacuum chamber, is used to gradually flow the labeled DNA through the channels of the wafer over the course of 30-60 minutes. The wafer is washed three times with hybridization buffer at 65° C.

Part E: Quantitation of Hybridization Signals

Following hybridization and washing, the wafer is briefly dried, then placed onto the phosphor screen of a phosphorimager and kept in the dark for a period of time determined by the intensity of label. The phosphor screen is then placed into the phosphorimager reader for quantitation of individual hybridization signals arising from each channel region in the array.

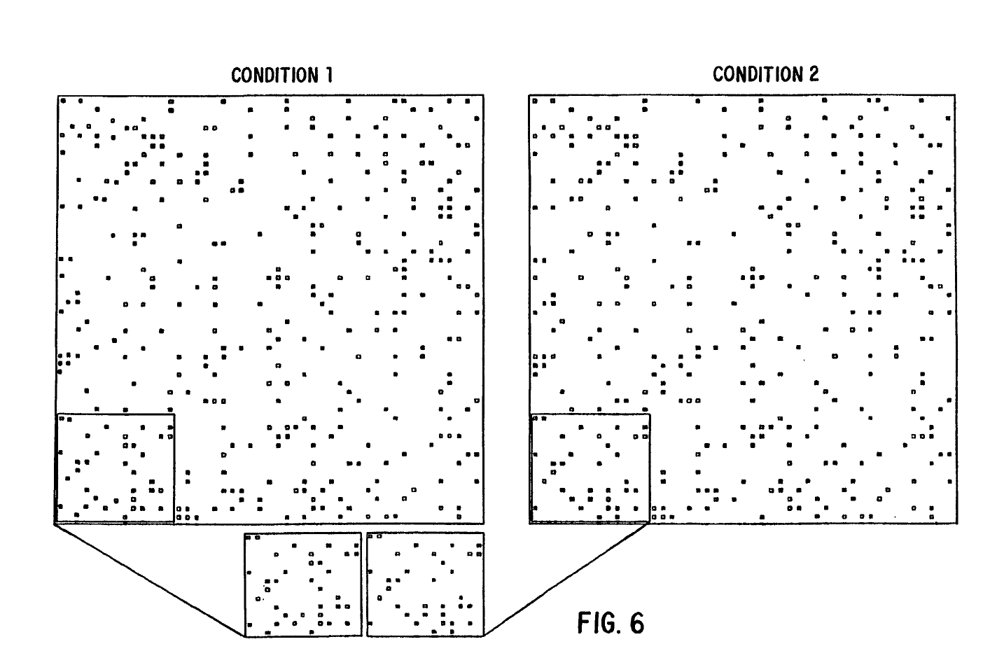

FIG. 6 illustrates results obtainable from a hybridization experiment. Total cytoplasmic mRNA is isolated from cells cultured under two conditions and subjected to the “differential display” procedure described above to prepare fragments representative of individual mRNA species present under the two conditions. These samples are hybridized to two identical cDNA arrays, to yield the two hybridization signal patterns shown. These patterns represent the profile of expressed genes under the two different culture conditions (for example in the presence and absence of a drug or chemical that induces a change in the expression of some genes). Note that overall, the pattern of hybridization is similar for the two conditions, but as expected for a differential expression of certain genes under the two conditions, there are a few hybridization signals that are seen only for culture condition 1 and a few that are seen only for culture condition 2. The box in the lower left, reproduced at the bottom of the figure to assist visual comparison, represents several differences in the gene expression profile. The squares represent sites where hybridization has occurred and the darkness of the squares is proportional to the number of labeled fragments present at each site.

The invention has been disclosed broadly and illustrated in reference to representative embodiments described above. Those skilled in the art will recognize that various modifications can be made to the present invention without departing from the spirit and scope thereof.

The disclosure of all publications cited above are expressly incorporated herein by reference in their entireties to the same extent as if each were incorporated by reference individually.

What is claimed is:

1. A device for binding a target molecule, comprising:
a substrate having oppositely facing first and second major surfaces;
a multiplicity of discrete channels extending through said substrate from said first major surface to said second major surface;
a first binding reagent immobilized in a first group of said channels, and
a second binding reagent immobilized in a second group of said channels.

2. A device according to claim 1, wherein said first and second binding reagents differ from one another.

3. A device according to claim 1, wherein said first and second binding reagents bind different target molecules.

4. A device according to claim 2, comprising discrete channels having diameters of from about 0.033 micrometers to about 10 micrometers.

5. A device according to claim 2, comprising discrete channels having cross sectional areas of between about 8.5×10⁻⁴ μm² to about 80 μm².

6. A device according to claim 2, comprising a substrate between about 100 μm to about 1000 μm thick.

7. A device according to claim 2, comprising channels having an inner surface area of between about 10 μm² and about 3×10⁶ μm².

8. A device according to claim 2, wherein said groups of channels have areas of between about 20 μm² to about 3×10⁶ μm².

9. A device according to claim 2, wherein there are between 400 and 4400 of said groups of discrete channels per cm² of cross-sectional area of the substrate.

10. A device according to claim 2, wherein the inner surface area of the channels in a group of channels is from about 100 to about 1000 times the cross sectional area of the group of channels.

11. A device according to claim 1, wherein said substrate is fabricated from glass or silicon.

12. A device according to claim 11, comprising a substrate made of nanochannel glass.

13. A device according to claim 12, comprising a substrate made of oriented array microporous silicon.

14. A device according to claim 1, wherein said binding reagents are effective for carrying out binding reactions selected from the group consisting of binding reactions involving small molecules, macromolecules, particles and cellular systems.

15. A device according to claim 14, wherein said binding reagents are effective for carrying out an analytical task selected from the group consisting of sequence analysis by hybridization, analysis of patterns of gene expression by hybridization of mRNA or cDNA to gene-specific probes, immunochemical analysis of protein mixtures, epitope mapping, assay of receptor-ligand interactions and profiling of cellular populations involving binding of cell surface molecules to specific ligands or receptors.

16. A device according to claim 15, wherein said binding reagents are selected from the group consisting of DNA, proteins and ligands.

17. A device according to claim 16, wherein said binding reagents are oligonucleotide probes.

18. A device according to claim 17, wherein the oligonucleotide probes are attached to channel surfaces via a primary amine group incorporated into the probes prior to immobilization.

19. A device according to claim 18, wherein said probes are attached to said channel surfaces via a terminal primary amine derivative of said polynucleotide and said glass substrate is derivatized with epoxysilane.

20. A device for binding a target molecule, comprising:
a substrate having oppositely facing first and second major surfaces;
a multiplicity of discrete channels extending through said substrate from said first major surface to said second major surface;
a first binding reagent immobilized in a first group of said channels, and
a second binding reagent immobilized in a second group of said channels,
further comprising a rigid support, wherein said rigid support is integral to said substrate, or is bonded to said substrate.

21. A device according to claim 20 wherein said support is integral to said substrate.

22. A device according to claim 20, wherein said support is bonded to said substrate.

23. A device according to claim 20, wherein said rigid support comprises wells for delivering fluids to subsets of channels of said substrate.

24. A device according to claim 20, comprising discrete channels having cross sectional areas of between about 8.5×10^{-4 μm}² to about 80 μm².

25. A device according to claim 20, comprising channels having an inner surface area of between about 10 μm² and about 3×10⁴ μm².

26. A device according to claim 20, wherein said groups of channels have areas of between about 20 μm² to about 3×10⁶ μm².

27. A device according to claim 20, wherein there are between 400 and 4400 of said discrete channels per cm² of cross-sectional area of the substrate.

28. A device according to claim 20, wherein the inner surface area of the channels in a group of channels is from about 100 to about 1000 times the cross sectional area of the group of channels.

29. A device according to claim 20, comprising a substrate fabricated from glass or silicon.

30. A device according to claim 29, comprising a substrate made of nanochannel glass.

31. A device according to claim 29, comprising a substrate made of oriented array microporous silicon.

32. A device according to claim 20, wherein said binding reagents are effective for carrying out binding reactions selected from the group consisting of binding reactions involving small molecules, macromolecules, particles and cellular systems.

33. A device according to claim 32, wherein said binding reagents are effective for carrying out an analytical task selected from the group consisting of sequence analysis by hybridization, analysis of patterns of gene expression by hybridization of mRNA or cDNA to gene-specific probes, immunochemical analysis of protein mixtures, epitope mapping, assay of receptor-ligand interactions and profiling of cellular populations involving binding of cell surface molecules to specific ligands or receptors.

34. A device according to claim 33, wherein said binding reagents are selected from the group consisting of DNA, proteins and ligands.

35. A device according to claim 34, wherein said binding reagents are oligonucleotide probes.

36. A device according to claim 35, wherein the oligonucleotide probes are attached to channel surfaces via a primary amine group incorporated into the probes prior to immobilization.

37. A device according to claim 36, wherein said probes are attached to said channel surfaces via a terminal primary amine derivative of said polynucleotide and said glass substrate is derivatized with epoxysilane.

38. A device according to claim 1, comprising discrete channels having diameters of from about 0.45 micrometers to about 10 micrometers.

39. A device according to claim 20, comprising discrete channels having diameters of from about 0.45 micrometers to about 10 micrometers.
